(19) United States
(12) Patent Application Publication
(10) Pub. No.: US 2020/0138358 A1
(43) Pub. Date: May 7, 2020

(54) ALZHEIMER'S DISEASE SYMPTOM EVALUATION SYSTEM

(71) Applicant: National Cheng Kung University, Tainan City (TW)

(72) Inventors: Pau-Choo CHUNG, Tainan City (TW); Ming-Chyi PAI, Tainan City (TW); Yu-Liang HSU, Taichung City (TW); Wei-Hsin Wang, Tainan City (TW)

(21) Appl. No.: 16/179,290

(22) Filed: Nov. 2, 2018

(51) Int. Cl. A61B 5/00; A61B 5/11

(52) U.S. Cl. CPC A61B 5/4088; A61B 5/4023; A61B 5/112; A61B 2562/0219; A61B 5/002; A61B 5/1121; A61B 5/6801

(57) ABSTRACT

An Alzheimer's disease symptom evaluation system includes at least one sensing device, for a subject to wear, the sensing device detecting body movement of the subject during a balance test and a gait test and recording as a plurality of movement informations; a processing unit and a cloud unit, the processing unit being adapted for connecting to the sensing device to read the movement informations and to upload them to the cloud unit, the cloud unit including a database unit and an analyzing unit, a plurality of symptom model informations being stored in the database unit, the uploaded movement informations being calculated and processed by the analyzing unit, the analyzing unit scoring the movement informations by comparing with the symptom model informations in the database unit to give a plurality of evaluation scores, the evaluation scores being scored overall to provide an evaluation information.

### BACKGROUND OF THE INVENTION

### Field of the Invention

The present invention relates to an Alzheimer's disease symptom evaluation system.

### Description of the Prior Art

There are 3.56 millions of Dementia patient in the world in 2015, and the number increases at 9.9 million per year to increase medical expenditure and pressure of patient's family. The commonest Dementia is Alzheimer's Disease which has an onset after age 65. The function of memory and cognition is declined over times, and independent ability is lost at the end. The process is irreversible.

To find out the onset of disease early to have an early treatment, mini-mental state examination and CASI (Cognitive Abilities Screening Instrument) are usually used to evaluate. The mini-mental state examination includes tests of arithmetic, memory, and sense of direction. The scores are summed up, and the result is judged by the intervals of total score. The CASI test includes tests of long-tern memory, short-term memory, orientation of time and place, attention, mental operation and concentration, fluency of thinking, language and basic cognition, abstract thinking and judgment, and eye-hand coordination and composition.

Besides, the disease can be evaluated by clinically observing the ability of balance and walking. BBS (Berg Balance Scale) is the most widely used scale. Traditionally, healthcare professionals record the time that the patient complete the movement, and the physical states such as difficulty of getting up, slow walking, and difficulty of turning back. The times of test are judged to provide a total score. However, the score is completely determined by the judgment of healthcare professionals to be subjective. Thus, some instruments provide objective data to healthcare professionals, such as in-shoe dynamic pressure measuring system “Pedar” by Novel, pressure pad system “Gait Mat II” by Tekscan. Those instruments measure the center of mass and gait by pressure measurement. However, the instruments are expensive and difficult to wear. Thus, small clinics or hospitals in remote areas are unable to afford.

### SUMMARY OF THE INVENTION

The main object of the present invention is to provide an Alzheimer's disease symptom evaluation system to provide evaluation quickly, conveniently, and economically.

To achieve the above and other objects, the Alzheimer's disease symptom evaluation system of the present invention includes at least one sensing device, a processing unit, and a cloud unit.

The at least one sensing device is for a subject to wear. The at least one sensing device detects body movement of the subject during a balance test and a gait test and records as a plurality of movement informations. The processing unit is adapted for connecting to the at least one sensing device to read the movement informations and to upload them to the cloud unit. The cloud unit includes a database unit and an analyzing unit. A plurality of symptom model informations are stored in the database unit. The uploaded movement informations are calculated and processed by the analyzing unit. The analyzing unit scores the movement informations by comparing with the symptom model informations in the database unit to give a plurality of evaluation scores. The evaluation scores are scored overall to provide an evaluation information.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is an illustration showing a system of the present invention;

FIG. 2 is a graph showing a result of balance test of the present invention;

FIG. 3 is an illustration showing a standing balance parameter of the present invention;

FIG. 4 is a graph showing a single step of the present invention;

FIG. 5 is a graph showing plural steps of the present invention;

FIG. 6 is a spectrogram showing step frequency of the present invention;

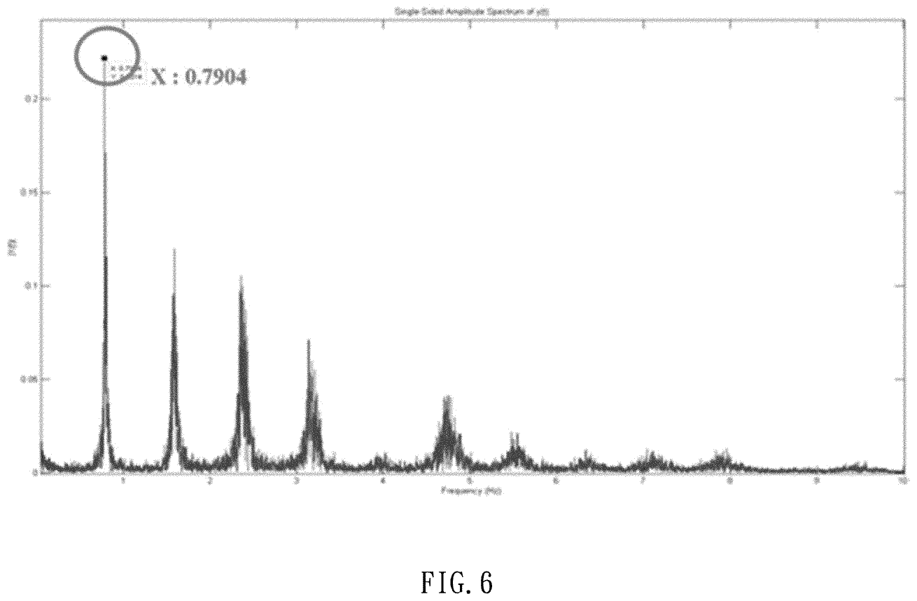

FIG. 7 is an illustration showing a stance phase and a swing phase of the present invention.

### DETAILED DESCRIPTION OF THE PREFERRED EMBODIMENTS

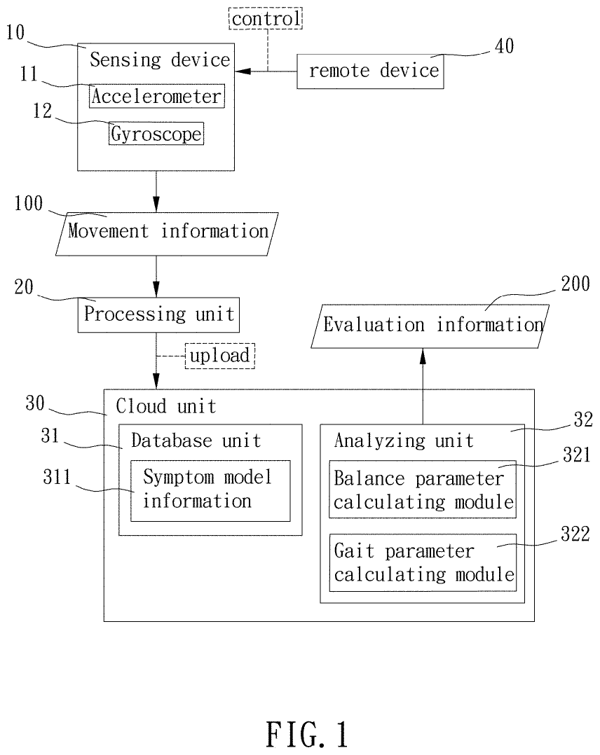

Please refer to FIG. 1, the Alzheimer's disease symptom evaluation system of the present invention includes at least one sensing device 10, a processing unit 20, and a cloud unit 30.

The at least one sensing device 10 is for a subject to wear. The at least one sensing device 10 detects body movement of the subject during a balance test and a gait test and records as a plurality of movement informations 100. The processing unit 20 is adapted for connecting to the at least one sensing device 10 to read the movement informations 100 and to upload them to the cloud unit 30. The cloud unit 30 includes a database unit 31 and an analyzing unit 32. A plurality of symptom model informations 311 are stored in the database unit 31. The uploaded movement informations 100 are calculated and processed by the analyzing unit 32. The analyzing unit 32 scores the movement informations 100 by comparing with the symptom model informations 311 in the database unit 31 to give a plurality of evaluation scores. The evaluation scores are scored overall to provide an evaluation information 200.

Specifically, the at least one sensing device 10 includes an accelerometer 11 or a gyroscope 12. The movement informations 100 are selected from a group composed of acceleration, velocity, displacement, angular velocity, and angle. Preferably, the system further includes a remote device 40. Each of the remote device 40 and the at least one sensing device 10 includes a wireless transmission unit. The remote device 40 is adapted for controlling the at least one sensing device 10 via the wireless transmission units. More preferably, the remote device 40 further includes a mobile electronic device and an application. The application is installed in the mobile electronic device to control the at least one sensing device 10. Each of the wireless transmission unit is an infrared transmission unit or a Bluetooth transmission unit.

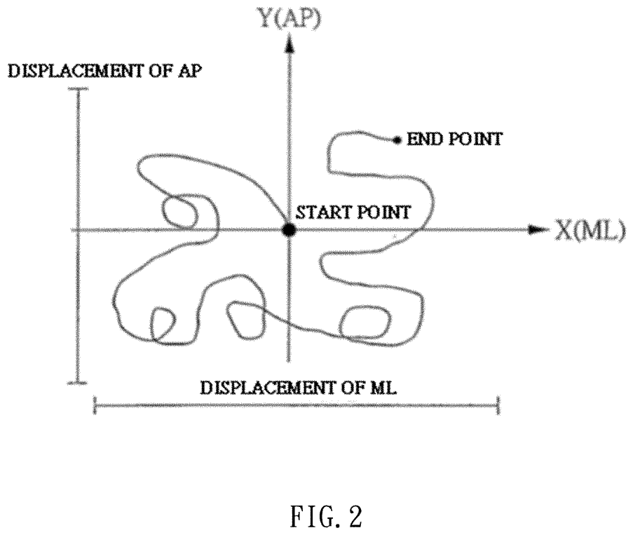

More specifically, during the balance test, the movement informations detected and recorded by the at least one sensing device 10 includes time of keeping balance, velocity along anterior-posterior (AP) axis, velocity along mediolateral (ML) axis, displacement along anterior-posterior (AP) axis, displacement along mediolateral (ML) axis, range of swing, and concentration of swing. The result is shown as a sample in FIG. 2. During the gait test, the movement informations detected and recorded by the at least one sensing device includes number of steps, time of walking, step length, step frequency, step regularity, step time, standing time in a step, swinging time in a step, variability of step regularity, variability of step symmetry, variability of step time, variability of standing time in a step, and variability of swinging time in a step. Practically, the analyzing unit 32 includes a balance parameter calculating module 321 and a gait parameter calculating module 322.

The following shows an embodiment of the present invention.

A. Balance test:

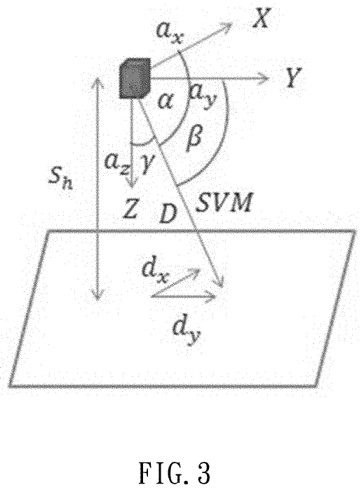

a. Standing balance test: The sensing device is worn by a subject at his waist, and the subject makes some movements, such as standing when opening or closing eyes, standing and aligning two feet along the AP axis when opening or closing eyes, and standing on one foot when opening or closing eyes. The balance time, velocity along the AP axis, and the velocity along the ML axis are obtained from the accelerometer, as shown in FIG. 3. The steps of calculation are shown below.

1. Calculate the signal vector magnitudes (SVMs) of acceleration along x, y, and z axis at each sampling point wherein aₓ, a_{y}, and a_{z} are the accelerations along x axis, y axis, and z axis at each sampling point.

SVM=\sqrt{αₓ²+α_{y}²+α_{z}²}

2. Calculate cosine of the acceleration along x axis, y axis, and z axis to SVMs.

cos α=αₓ/SVM

cos β=α_{y}/SVM

cos γ=α_{z}/SVM

3. Calculate the path distance (D) projected by the sensing device. The path distance is the ratio of distance between the sensing device and the ground to the cosine along z axis.

D =Sₓ/cos γ

4. Calculate the displacement (dₓ) on the ground along the AP axis from the path distance (D) and the cosine along x axis.

dₓ=D cos α

5. Calculate the displacement (d_{y}) on the ground along the ML axis from the path distance (D) and the cosine along y axis.

d_{y}=D cos β

Thereby, time of keeping balance (sec), velocity along AP axis (cm/sec), and velocity along ML axis (cm/sec) are obtained. The time of keeping balance is the time that the subject keeps balance during the balance test. The velocity along AP axis is the displacement (dₓ) along the AP axis per second in every test. The velocity along ML axis is the displacement (d_{y}) along the ML axis per second in every test.

b. Timed Up-and-Go test:

The subject sits on the chair at first, and then stands up to walk to a wall 3 m away straightly without touching the wall. And then, the subject turns back and walks back to the starting point. And then, the subject turns back again to sit down. The elapsed time of the test is recorded.

B. Gait test:

The subject walks for 40 m at a single occurrence (without other interference) or at double occurrence (such as counting down from 100 at the same time). The gait informations are collected by the sensing devices on two insteps. The steps of calculations are shown below.

1. Calculate the SVMs of acceleration and SVMs of angular velocity at each sampling point. aₓ, a_{y}, and a_{z} are acceleration along x axis, y axis, and z axis at each sampling point. ωₓ, ω_{y}, and ω_{z} are angular velocity along x axis, y axis, and z axis at each sampling point. SVMₐ and SVM_{ω}are SVMs of acceleration and angular velocity respectively.

SVM_{α}=\sqrt{αₓ²+α_{y}²+α_{z}²}

SVM₁₀₇ =\sqrt{ωₓ²+ω_{y}²+ω_{z}²}

2. Set the range of window as three sampling points, and calculate the variability of SVMₐ and the variability of SVM_{ω}.

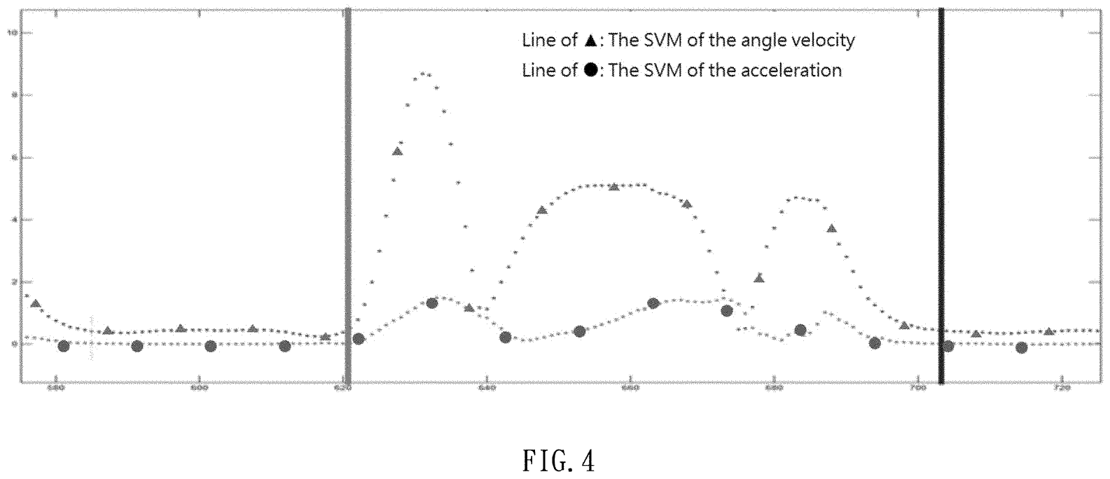

3. When the variability of SVMₐ is greater than 0.001 and the variability of SVM_{ω}is greater than 0.1, the first sampling point in the window is defined as the start point of the step, as shown as the line with circles in FIG. 4.

4. When the variability of SVMₐ is smaller than 0.0005, the variability of SVM_{ω}is greater than 0.0005, and a difference between SVMₐ and SVM_{ω}, is smaller than 10 times a difference between SVMₐ and SVM_{ω}, during motionless state, the first sampling point is defined as the end point of the step, as shown as the line with triangles in FIG. 4.

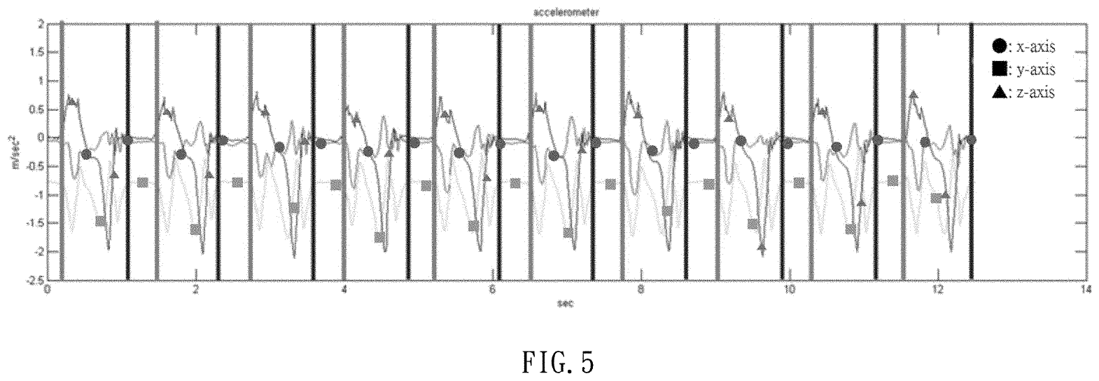

5. Repeat step 1 to step 4 to find the start point and end point of all steps during the 40 m-walking in order to calculate the step number, as shown in FIG. 5.

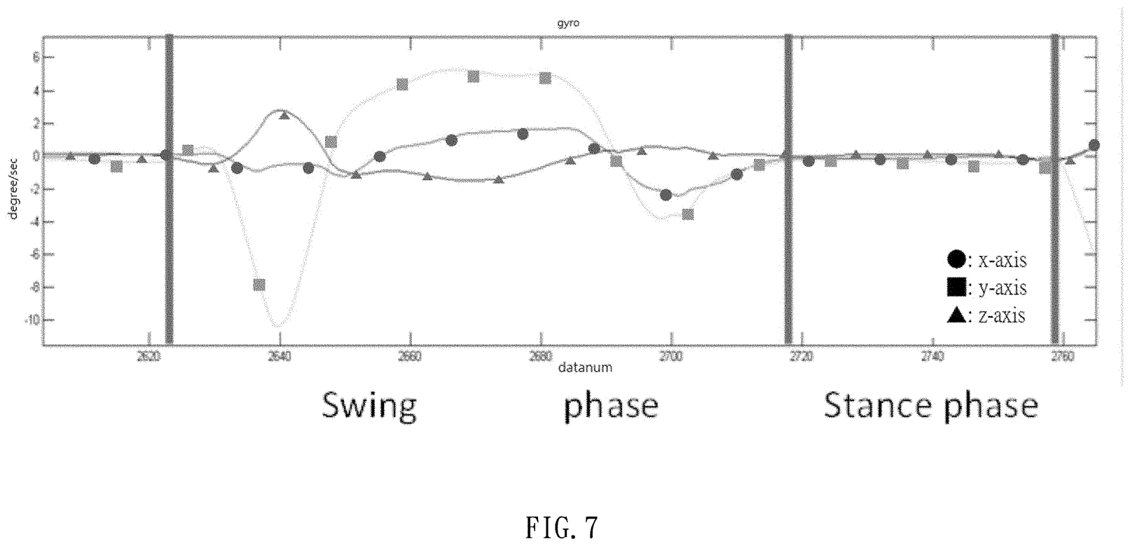

Thereby, the following parameters can be obtained: (1) Number of steps: How many steps the subject used to complete the 40 m-walking is obtained by the step-sensing method; (2) Time of walking: How much time the subject spent to complete the 40 m-walking is obtained by the step-sensing method; (3) Step length: Calculated by 40 m/number of steps; (4) Step frequency: Inertia sensing signals in time domain are transferred into signals in frequency domain by the gait parameter calculating module using Fourier transform, and the step frequency is obtained by designating main signals; (5) Step velocity: The step length multiply the step frequency is the step velocity; (6) Step rhythm: Step rhythm is obtained by number of steps/walking time; (7) Step regularity: Evaluated from the acceleration signals at a single foot; (8) Step symmetry: Evaluated from the acceleration signals at two feet; (9) Step time: The time between the start point and the end point in every step. Every step is partitioned into a stance phase and a swing phase, as shown in FIG. 7; (10) Standing time in a step: The time between the start point and the end point in the stance phase; (11) Swinging time in a step: The time between the start point and the end point in the swing phase; (12) Variability of step regularity: Calculated by the ratio of standard deviation of step regularity to the mean of step regularity multiplying 100%; (13) Variability of step symmetry: Calculated by the ratio of standard deviation of step symmetry to the mean of step symmetry multiplying 100%; (14) Variability of step time: Calculated by the ratio of standard deviation of step time to the mean of step time multiplying 100%; (15) Variability of standing time in a step: Calculated by the ratio of standard deviation of standing time in a step to the mean of standing time in a step multiplying 100% ; (16) Variability of swinging time in a step: Calculated by the ratio of standard deviation of swinging time in a step to the mean of swinging time in a step multiplying 100%.

The movement informatons are uploaded to the cloud unit, and the gait parameter calculating module calculates the parameters above. The parameters obtained by the balance parameter calculating module and the gait parameter calculating module are compared to the symptom model informations in the database unit and are scored respectively. All the scores are further summed up to be a total score. The total score is evaluated to provide an evaluation information showing the similarity between the subject and Alzheimer's disease patient. Thus, the possibility and level of Alzheimer's disease can be surmised.

In conclusion, the system of the present invention can provide an objective, accurate, and comprehensive evaluation information of Alzheimer's disease at lower cost and simple equipment. Alzheimer's disease can be easily diagnosed, and patients are willing to receive the test.

What is claimed is:

1. An Alzheimer's disease symptom evaluation system, including:
at least one sensing device, for a subject to wear, the at least one sensing device detecting body movement of the subject during a balance test and a gait test and recording as a plurality of movement informations;
a processing unit and a cloud unit, the processing unit being adapted for connecting to the at least one sensing device to read the movement informations and to upload them to the cloud unit, the cloud unit including a database unit and an analyzing unit, a plurality of symptom model informations being stored in the database unit, the uploaded movement informations being calculated and processed by the analyzing unit, the analyzing unit scoring the movement informations by comparing with the symptom model informations in the database unit to give a plurality of evaluation scores, the evaluation scores being scored overall to provide an evaluation information.

2. The Alzheimer's disease symptom evaluation system of claim 1, wherein the at least one sensing device includes an accelerometer or a gyroscope.

3. The Alzheimer's disease symptom evaluation system of claim 2, wherein the movement informations are selected from a group composed of acceleration, velocity, displacement, angular velocity, and angle.

4. The Alzheimer's disease symptom evaluation system of claim 1, wherein during the balance test, the movement informations detected and recorded by the at least one sensing device includes at least one in a group composed of time of keeping balance, velocity along anterior-posterior axis, velocity along mediolateral axis, displacement along anterior-posterior axis, displacement along mediolateral axis, range of swing, and concentration of swing.

5. The Alzheimer's disease symptom evaluation system of claim 2, wherein during the gait test, the movement informations detected and recorded by the at least one sensing device includes at least one in a group composed of number of steps, time of walking, step length, step frequency, step regularity, step time, standing time in a step, swinging time in a step, variability of step regularity, variability of step symmetry, variability of step time, variability of standing time in a step, and variability of swinging time in a step.

6. The Alzheimer's disease symptom evaluation system of claim 1, further including a remote device, each of the remote device and the at least one sensing device including a wireless transmission unit, the remote device being adapted for controlling the at least one sensing device via the wireless transmission units.

7. The Alzheimer's disease symptom evaluation system of claim 6, wherein the remote device further includes a mobile electronic device and an application, the application is installed in the mobile electronic device to control the at least one sensing device, each of the wireless transmission unit is an infrared transmission unit or a Bluetooth transmission unit.

8. The Alzheimer's disease symptom evaluation system of claim 2, wherein the analyzing unit includes a balance parameter calculating module and a gait parameter calculating module.

9. The Alzheimer's disease symptom evaluation system of claim 8, wherein inertia sensing signals in time domain are transferred into signals in frequency domain by the gait parameter calculating module using Fourier transform, the step frequency is obtained by designating main signals.

10. The Alzheimer's disease symptom evaluation system of claim 8, wherein a window is defined as three sampling points by the gait parameter calculating module, an variability of signal vector magnitude (SVM) of acceleration and an variability of signal vector magnitude (SVM) angular velocity are obtained by the gait parameter calculating module; a first sampling point is defined as a start point of step when the variability of SVM of acceleration is greater than 0.001 and the variability of SVM of angular velocity is greater than 0.1; a first sampling point is defined as an end point of step when the variability of SVM of acceleration is smaller than 0.0005, the variability of SVM of angular velocity is greater than 0.0005, and a difference between the SVM of acceleration and the SVM of angular velocity is smaller than 10 times a difference between the SVM of acceleration and the SVM of angular velocity during motionless state.
